(19) United States
(12) Patent Application Publication
(10) Pub. No.: US 2015/0282977 A1
(43) Pub. Date: Oct. 8, 2015

(54) Erection Management System

(71) Applicant: Tillman Wade Branham, SR., Gaston, SC (US)

(72) Inventor: Tillman Wade Branham, SR., Gaston, SC (US)

(21) Appl. No.: 14/680,060

(22) Filed: Apr. 6, 2015

Related U.S. Application Data
(60) Provisional application No. 61/975,252, filed on Apr. 4, 2014.

(51) Int. Cl. A61F 5/41; A61H 19/00; A61H 7/00

(52) U.S. Cl. CPC A61F 5/41; A61H 7/001; A61H 19/32; A61F 2005/412

(57) ABSTRACT

An erection management system utilizes a vacuum tube assembly to achieve an erection, and a tension band assembly to maintain the erection. The vacuum tube assembly is collapsible, with a first tube and a second tube being telescopically engaged. A conical diaphragm within the first and second tube separate an erection chamber and a vacuum chamber. Vacuum can be applied to the vacuum chamber to expand the diaphragm and thereby the penis to achieve an erection. A flexible tube of the tension band assembly is criss-crosses through a restrictor sleeve, and a tension loop is tightened around the base of the penis by pulling on opposite handles of the flexible tube. A tension locking mechanism is used to secure tension on the tension loop and a release cord is used to release the tension.

The current application claims a priority to the U.S. Provisional Patent application Ser. No. 61/975,252 filed on Apr. 4, 2014. The current application is filed on Apr. 6, 2015, whereas Apr. 4, 2015 and Apr. 5, 2015 were on a weekend.

### FIELD OF THE INVENTION

The present invention relates generally to sexual performance. More specifically, the present invention relates to an apparatus for erection management.

### BACKGROUND OF THE INVENTION

Human sexual activity has cognitive, emotional, behavioral and biological aspects; these include personal bonding, sharing emotions, and the physiology of the reproductive system, sex drive, sexual intercourse and sexual behavior in all its forms. Sexual intercourse, also known as coitus or copulation, in humans, typically requires the penis of the male to be in an erect state, wherein the penis is pressurized by being filled with blood. While penile-vaginal penetration is typically the definition of sexual intercourse, other types of sexual activity exist such as foreplay including manual or oral stimulation of the genitals.

Erectile dysfunction (ED) or impotence is a sexual dysfunction characterized by the inability to develop or maintain an erection of the penis during sexual activity. A penile erection is the hydraulic effect of blood entering and being retained in sponge-like bodies within the penis. The most important organic causes are cardiovascular disease and diabetes, neurological problems (for example, trauma from prostatectomy surgery), hormonal insufficiencies (hypogonadism) and drug side effects. Treatments vary, from exercise, to oral medication, to topical or injected medication, surgery, and penis pumps.

A penis pump is a cylinder that is fitted over the penis, with a manual or motorized pump to create a partial vacuum around the penis, engorging it as blood is drawn in. A variety of penis pump designs have been adopted by penis pump manufacturers. As vacuum increases, the pressure within the blood vessels of the penis increases as well. Penis pumps may be used to temporarily overcome symptoms of impotence, but do not cure the condition: a tourniquet-like ring must be applied to keep the blood trapped within the penis, otherwise the erection will immediately subside.

It is an object of the present invention to provide an erection management system which is easily portable, easy to use, and effective.

### BRIEF DESCRIPTION OF THE DRAWINGS

FIG. 1 is a side cross-sectional view of the vacuum tube assembly.

FIG. 2 is a rear perspective view of the vacuum tube assembly.

FIG. 3 is a rear perspective exploded view of the vacuum tube assembly.

FIG. 4 is a side view of the vacuum tube assembly with attached vacuum applicator tube and vacuum indicator portion.

FIG. 5 is a perspective view of the tension band assembly.

FIG. 6 is a perspective view of the tension band assembly with the elastic locking band removed.

FIG. 7 is a side perspective view of the tension band assembly.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced without the implementation of some features as they are described. It should be understood that some details have not been described in detail in order to not unnecessarily obscure focus of the invention.

The present invention is an erection management system comprising two primary components—a vacuum tube assembly 1 and a tension band assembly 2. The vacuum tube assembly 1 is used to manually produce an erection in the user, while the tension band assembly 2 is used to constrict the penis of the user in order to maintain the erection.

In the preferred embodiment of the present invention, the vacuum assembly is collapsible in order to be easily portable. It is contemplated that the vacuum tube assembly 1 does not need to be collapsible for the present invention to function properly, however the collapsing capability of the vacuum tube assembly 1 is desired in the preferred embodiment.

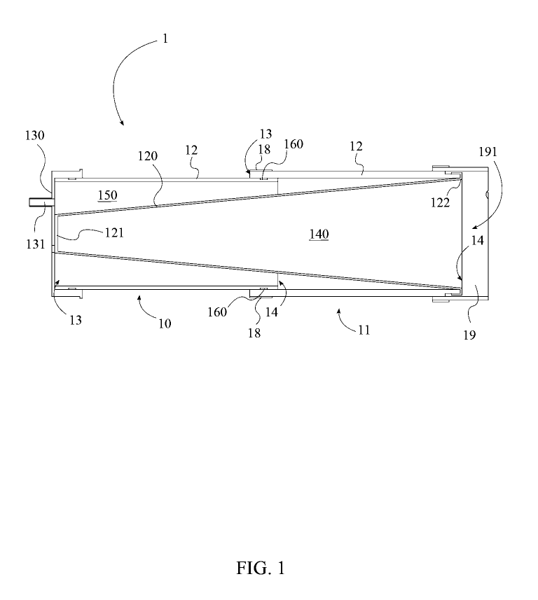

Referring to FIG. 1, in the preferred embodiment of the present invention, the vacuum tube assembly 1 comprises a first tube 10, a second tube 11, a conical diaphragm 120, and an end cap 130. The first tube 10 and the second tube 11 each comprise a lateral wall 12 which is cylindrical, a first end 13 and a second end 14. It is contemplated that the lateral wall 12 may not necessarily need to be cylindrical, and may be another shape, such as, but not limited to, rectangular and cuboidal, so long as the shape does not impede the functionality of the vacuum tube assembly 1. The first end 13 and the second end 14 are defined in order to subsequently explain relations between components. The first end 13 is defined as the end positioned away from the user while in use, and the second end 14 is defined as the end positioned closer to the user while in use. For example, when the vacuum tube assembly 1 is extended and in use by a user to create an erection, the second end 14 of the second tube 11 is positioned immediately adjacent to the groin of the user, while the first end 13 of the first end 13 is separated from the user by the majority of the vacuum tube assembly 1. Thus, the first end 13 of the first tube 10 is positioned opposite the second end 14 of the second tube 11 along the vacuum tube assembly 1.

In the preferred embodiment, the first tube 10 is telescopically engaged within the second tube 11. Thus, the outer diameter of the lateral wall 12 of the first tube 10 is approximately equal to the inner diameter of the lateral wall 12 of the second tube 11, with a minimal clearance.

The end cap 130 is connected to the first end 13 of the first tube 10. The end cap 130 functions to seal the vacuum tube assembly 1 at the first end 13 of the first tube 10. The conical diaphragm 120 is positioned within the first tube 10 and the second tube 11. The conical diaphragm 120 is made of a flexible material which allows the conical diaphragm 120 to expand and collapse in response to changes in air pressure within the first tube 10 and the second tube 11. The conical diaphragm 120 comprises an apex 121 and a base rim 122 which are positioned opposite each other along the conical diaphragm 120. The apex 121 is connected to the end cap 130, and the base rim 122 is perimetrically connected to the second end 14 of the second tube 11. The result is that an erection chamber 140 is delineated within the conical diaphragm 120, and a vacuum chamber 150 is delineated between the conical diaphragm 120 and the lateral walls 12 of the first tube 10 and the second tube 11. It is important to the functionality of the present invention that the erection chamber 140 and the vacuum chamber 150 are separated from each other and properly sealed. Finally, a vacuum port 131 traverses through the end cap 130 into the vacuum chamber 150. The vacuum port 131 allows fluid communication with an air pumping means in order to manipulate the air pressure within the vacuum chamber 150. Additionally, it is contemplated that the apex 121 of the conical diaphragm 120 may not necessarily need to be connected to the end cap 130, and may simply be free-floating within the vacuum chamber 150. The conical diaphragm 120 should also not be specifically limited to being conical in shape, and may take any other shape which may support the intended functionality of the present invention.

It is contemplated that multiple means of manipulating the telescoping action of the first tube 10 and the second tube 11 with each other may be utilized. The following is not intended to limit the functionality of the telescoping action and locking of the vacuum tube assembly 1, but is a description of the components and functionality of the preferred embodiment of the present invention.

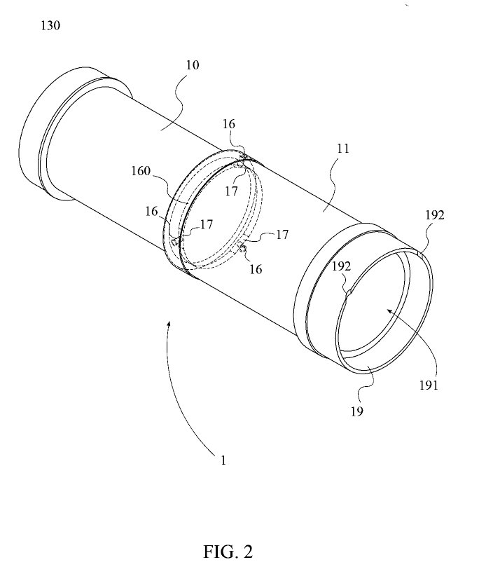

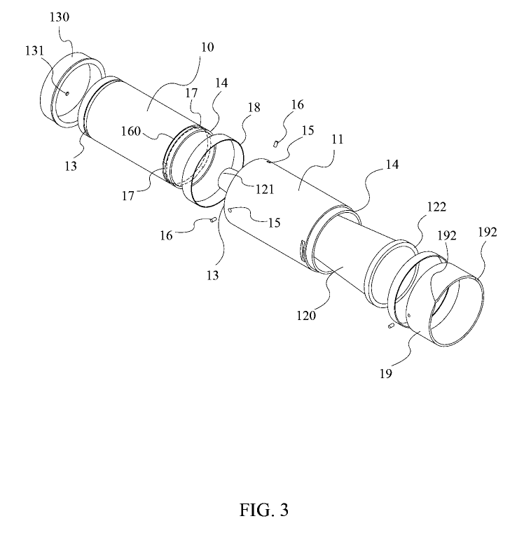

Referring to FIGS. 1-3, an annular groove 160 traverses into the lateral wall 12 adjacent to the second end 14 of the first tube 10. It is important to note that the annular groove 160 traverses from the outer wall of the lateral wall 12 of the first tube 10 inward toward the inner wall of the lateral wall 12 of the first tube 10. At least one pin locking pin slot traverses from the annular groove 160 into the lateral wall 12, offset from the vacuum chamber 150. The second tube 11 comprises at least one pin hole 15 and at least one locking pin 16. The pin holes 15 are radially distributed around the lateral wall 12 of the second tube 11. Each of the locking pins 16 is positioned within one of the pin holes 15. In order to lock the vacuum tube assembly 1 into the extended position, the first tube 10 is drawn outward from within the second tube 11 until the locking pins 16 fall into the annular groove 160. The first tube 10 and the second tube 11 are locked axially in place by rotating the first tube 10 and the second tube 11 relative to each other until the locking pins 16 fall into the pin locking slots 17. In the preferred embodiment, the pin locking slots 17 are crescent shaped, so that in order to disengage the first tube 10 and the second tube 11 from the locked position, the first tube 10 and the second tube 11 are rotated relative to each other, allowing the locking pins 16 to ramp up the crescent shape of the pin locking slots 17 until the annular groove 160 is reached. Additionally in the preferred embodiment, an annular elastic band 18 encircles the second tube 11 adjacent to the pin holes 15 so that the annular elastic band 18 holds the locking pins 16 within the pin holes 15 and provides the force necessary to push the locking pins 16 into the pin holes 15. It is contemplated however that this may be accomplished through alternate means, such as, but not limited to, springs connected to each of the locking pins 16.

In the preferred embodiment, a base cap 19 is perimetrically connected to the second end 14 of the second tube 11, with an erection aperture 191 traversing through the base cap 19 into the erection chamber 140. When in use, the user will insert the penis through the erection aperture 191 into the erection chamber 140, pressing the base cap 19 against their groin to properly seal the erection chamber 140. Additionally, the base cap 19 comprises a plurality of tension band accepting slots 192, which are positioned on the base cap 19 opposite the erection chamber 140 and positioned laterally equidistant from each other, or in any other configuration necessary to accept the tension band while in use. The tension band accepting slots 192 provide a space for the tension band while the present invention is in use in order to facilitate a proper seal at the base cap 19.

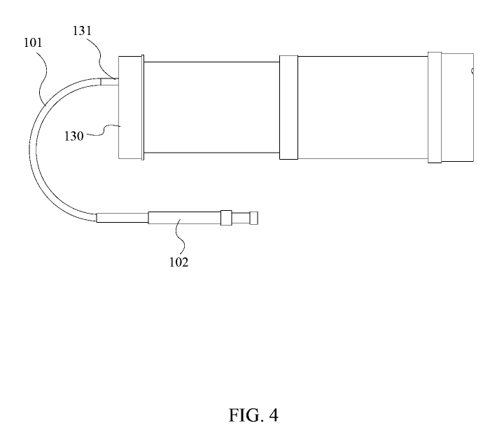

An additional component comprised in the preferred embodiment of the present invention is a vacuum applicator tube 101 as shown in FIG. 4. The vacuum applicator tube 101 is preferably removably attached to the vacuum port 131 on the end cap 130. It is contemplated that the vacuum applicator tube 101 may be permanently affixed to the vacuum port 131, though this is not ideal for portability and convenience. In one embodiment, the vacuum applicator tube 101 may be a simple flexible tube 21, allowing the user to orally apply suction to the vacuum chamber 150 through the vacuum applicator tube 101. However, in the preferred embodiment, it is desired to provide a means for indicating when sufficient vacuum has been applied to the vacuum chamber 150. It is contemplated that a number of means may be utilized to this end, but in the preferred embodiment, the vacuum applicator tube 101 comprises a vacuum indicator portion 102. The vacuum indicator portion 102 is in fluid communication with the vacuum port 131 through the vacuum applicator tube 101, and is made of a flexible material. The result is that when sufficient vacuum has been reached, the vacuum indicator portion 102 collapses when a suitable vacuum threshold is reached within the vacuum chamber 150. This provides an indicator to the user that is not only visual but also tactile, so that the user may ascertain whether sufficient vacuum has been reached in low lighting conditions by touching the vacuum indicator portion 102 and feeling whether the vacuum indicator portion 102 is collapsed or not. Collapse of the vacuum indicator portion 102 additionally functions to occlude the vacuum applicator tube 101 so that when the suitable vacuum threshold is reached, no further vacuum may be applied. In an alternate embodiment, the vacuum indicator portion 102 may be directly connected to the end cap 130 and thus be in fluid communication with the vacuum chamber 150 through a second vacuum port 131, while the user applies vacuum through the vacuum port 131. Additionally, one embodiment of the present invention may include two vacuum ports 131, so that the user may utilize a combination of manual oral application of vacuum through one of the ports, while an electrical or mechanical device is utilized to apply vacuum through the other port.

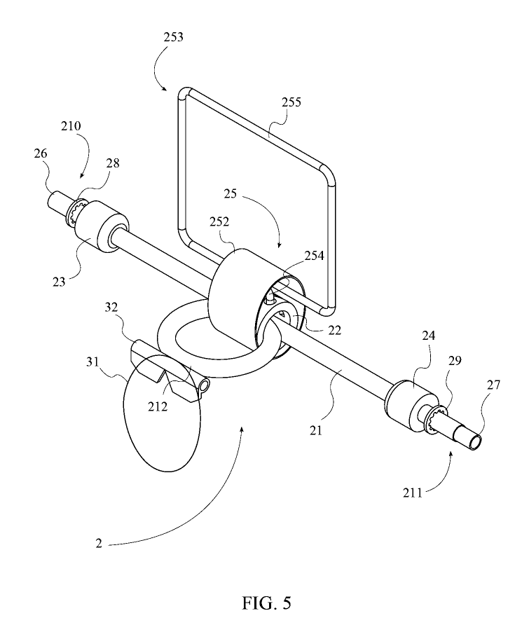

Referring to FIG. 5, the tension band assembly 2 comprises a flexible tube 21, a restrictor sleeve 22, a first pull handle 23, a second pull handle 24, and a tension locking mechanism 25. A first end 210 and a second end 211 of the flexible tube 21 are positioned opposite each other along the flexible tube 21 at the extremes of the flexible tube 21. The first pull handle 23 is connected to the first end 210 of the flexible tube 21, and the second pull handle 24 is connected to the second end 211 of the flexible tube 21. Furthermore, the first pull handle 23 and the second pull handle 24 encircle the flexible tube 21 in the preferred embodiment.

The flexible tube 21 is internally criss-crossed through the restrictor sleeve 22 so that the flexible tube 21 forms a tension loop 212 between the first end 210 and the second end 211 that passes through the restrictor sleeve 22. The tension locking mechanism 25 is removably engaged with the tension loop 212, wherein the tension locking mechanism 25 functions to maintain and release tension on the tension loop 212 within the restrictor sleeve 22.

The tension band assembly 2 further comprises a first end plug 26, a second end plug 27, a first lock washer 28, and a second lock washer 29. The first end plug 26 is inserted within the first end 210 of the flexible tube 21, and the second end plug 27 is inserted within the second end 211 of the flexible tube 21. The first end plug 26 and the second end plug 27 should be hollow in order to ensure that the flexible tube 21 is not sealed. The first lock washer 28 and the second lock washer 29 should be inside tooth washers. The first lock washer 28 is engaged with the flexible tube 21 adjacent to the first pull handle 23, and the second lock washer 29 is engaged with the flexible tube 21 adjacent to the second pull handle 24. More specifically, the first lock washer 28 encircles the flexible tube 21 at the first end 210, and the second lock washer 29 encircles the flexible tube 21 at the second end 211. The first lock washer 28 is positioned between the first pull handle 23 and the first end plug 26. Similarly, the second lock washer 29 is positioned between the second pull handle 24 and the second end plug 27.

In one embodiment of the present invention, the tension locking mechanism 25 comprises a tensioning pin 251, an elastic locking band 252, and a release cord 253. In this embodiment, the restrictor sleeve 22 comprises a tension pin hole 223 which laterally traverses into the restrictor sleeve 22. The tensioning pin 251 is slidably engaged within the tension pin hole 223. The elastic locking band 252 is positioned around the restrictor sleeve 22 adjacent to the tension pin hole 223, so that the elastic locking band 252 applies pressure to the tensioning pin 251. The release cord 253 is connected to the tensioning pin 251. The result of this setup is that the tensioning pin 251 is constantly applying pressure to a portion of the tension loop 212 within the restrictor sleeve 22. When the user pulls the first pull handle 23 and the second pull handle 24 apart and subsequently releases the pull handles, the tensioning pin 251 automatically is applying pressure to the tension loop 212, allowing the tension loop 212 to retain the tension applied by pulling the ends of the flexible tube 21 apart from each other. In order to release the tensioning mechanism, the user pulls on the release cord 253, which overcomes the elastic force applied by the tension band, pulling the tensioning pin 251 away from the tension loop 212 and thus releasing the tension on the tension loop 212.

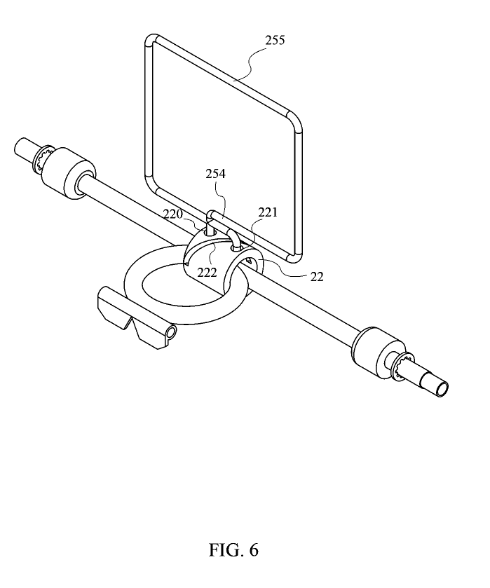

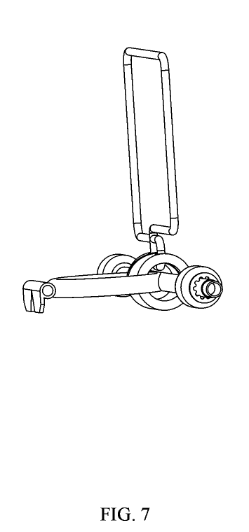

In another embodiment shown in FIGS. 5-7, the tension locking mechanism 25 comprises an elastic locking band 252, a release cord 253, a first cord hole 220 and a second cord hole 221 of the restrictor sleeve 22, and a diagonal slot 222 of the restrictor sleeve 22. The diagonal slot 222 traverses into the restrictor sleeve 22 diagonally from a first end 13 of the restrictor sleeve 22 to a second end 14 of the restrictor sleeve 22. The diagonal slot 222 penetrates entirely through the wall of the restrictor sleeve 22. The first cord hole 220 and the second cord hole 221 are positioned opposite each other across the diagonal slot 222. The release cord 253 traverses through the first cord hole 220 and the second cord hole 221. The release cord 253 comprises a criss-cross portion 254 and a pull portion 255. The release cord 253 crosses over itself at the criss-cross portion 254 so that the release cord 253 forms a loop around the diagonal slot 222 at the criss-cross portion 254, which can be seen in FIGS. 6-7. The elastic locking band 252 is positioned around the restrictor sleeve 22 and the criss-cross portion 254 opposite the flexible tube 21, so that the restrictor sleeve 22 applies lateral pressure to the restrictor sleeve 22. This causes the restrictor sleeve 22 to be deformed due to the lack of structural integrity from the existence of the diagonal slot 222. The pull portion 255 is positioned adjacent to the elastic locking band 252 opposite the criss-cross portion 254. After the tension loop 212 is tightened, in order to release tension on the tension loop 212 the user applies force to the pull portion 255 of the release cord 253 away from the restrictor sleeve 22. This causes the release cord 253 to tighten around the diagonal slot 222 with the criss-cross portion 254, causing the restrictor sleeve 22 to deform since the first cord hole 220 and the second cord hole 221 are forced toward each other. The walls of the restrictor sleeve 22 on either side of the diagonal slot 222 collapse toward each other, acting like cams. One of said walls rides up over the other, deforming the restrictor sleeve 22 enough to allow the tension loop 212 to release tension so that the user may remove the tension band assembly 2 from the base of the penis.

The preferred embodiment of the present invention additionally comprises a comfort strip 30 which is externally connected to the restrictor sleeve 22. Additionally, the tension band assembly 2 further comprises a testicle band 31 which is connected tangent to the tension loop 212 opposite the restrictor sleeve 22. The tension band assembly 2 also further comprises a sperm release cradle 32, which is connected to the tension loop 212 opposite the restrictor sleeve 22.

Although the invention has been explained in relation to its preferred embodiment, it is to be understood that many other possible modifications and variations can be made without departing from the spirit and scope of the invention as hereinafter claimed.

What is claimed is:

1. An erection management system comprises:
a vacuum tube assembly;
a tension band assembly;
the vacuum tube assembly comprises a first tube, a second tube, a conical diaphragm, and an end cap;
the first tube and the second tube each comprise a lateral wall, a first end, and a second end;
the first tube being telescopically engaged within the second tube;
the first end of the first tube being positioned opposite the second end of the second tube along the vacuum tube assembly;
the end cap being connected to the first end of the first tube;
the conical diaphragm being positioned within the first tube and the second tube;
the conical diaphragm comprises an apex and a base rim;
the apex and the base rim being positioned opposite each other along the conical diaphragm;
the apex being connected to the end cap;
the base rim being perimetrically connected to the second end of the second tube;
an erection chamber being delineated within the conical diaphragm;
a vacuum chamber being delineated between the conical diaphragm and the lateral walls of the first tube and the second tube;
a vacuum port traversing through the end cap into the vacuum chamber;
the tension band assembly comprises a flexible tube, a restrictor sleeve, a first pull handle, a second pull handle, and a tension locking mechanism;
a first end and a second end of the flexible tube being positioned opposite each other along the flexible tube;
the first pull handle being connected to the first end of the flexible tube;
the second pull handle being connected to the second end of the flexible tube;
the flexible tube being internally criss-crossed through the restrictor sleeve, forming a tension loop between the first end and the second end passing through the restrictor sleeve; and
the tension locking mechanism being removably engaged with the tension loop, wherein the tension locking mechanism maintains and releases tension on the tension loop.

2. The erection management system as claimed in claim 1 comprises:
an annular groove traversing into the lateral wall adjacent to the second end of the first tube;
at least one pin locking slot traversing from the annular groove into the lateral wall offset from the vacuum chamber;
the second tube comprises at least one pin hole and at least one locking pin;
the pin holes being radially distributed around the lateral wall of the second tube; and
each of the locking pins being positioned within one of the pin holes.

3. The erection management system as claimed in claim 2 comprises:
each of the pin locking slots being crescent shaped.

4. The erection management system as claimed in claim 2 comprises:
an annular elastic band encircling the second tube adjacent to the pin holes, wherein the annular elastic band holds the locking pins within the pin holes.

5. The erection management system as claimed in claim 1 comprises:
a base cap being perimetrically connected to the second end of the second tube; and
an erection aperture traversing through the base cap into the erection chamber.

6. The erection management system as claimed in claim 5 comprises:
the base cap comprises a plurality of tension band accepting slots;
the tension band accepting slots being positioned on the base cap opposite the erection chamber; and
the band accepting slots being laterally equidistant from each other.

7. The erection management system as claimed in claim 1 comprises:
the vacuum tube assembly further comprises a vacuum applicator tube; and
the vacuum applicator tube being removably attached to the vacuum port on the end cap.

8. The erection management system as claimed in claim 7 comprises:
the vacuum applicator tube comprises a vacuum indicator portion;
the vacuum indicator portion being in fluid communicate on with the vacuum port; and
the vacuum indicator portion being made of a flexible material, wherein the vacuum indicator portion collapses when a suitable vacuum threshold is reached within the vacuum chamber.

9. The erection management system as claimed in claim 1 comprises:
the tension band assembly further comprises a first end plug, a second end plug, a first lock washer, and a second lock washer—inside tooth washers;
the first end plug being inserted within the first end of the flexible tube;
the second end plug being inserted within the second end of the flexible tube;
the first lock washer being engaged with the flexible tube adjacent to the first pull handle; and
the second lock washer being engaged with the flexible tube adjacent to the second pull handle.

10. The erection management system as claimed in claim 1 comprises:
the tension locking mechanism comprises a tensioning pin, an elastic locking band, and a release cord;
the restrictor sleeve comprises a tension pin hole;
the tension pin hole laterally traversing into the restrictor sleeve;
the tensioning pin being slidably engaged within the tension pin hole;
the elastic locking band being positioned around the restrictor sleeve adjacent to the tension pin hole; and
the pull string being connected to the tensioning pin.

11. The erection management system as claimed in claim 1 comprises:
the tension locking mechanism comprises an elastic locking band, a release cord, a first cord hole and a second cord hole of the restrictor sleeve, and a diagonal slot of the restrictor sleeve;
the diagonal slot traversing into the restrictor sleeve;
the diagonal slot traversing from a first end of the restrictor sleeve to a second end of the restrictor sleeve;
the first cord hole and the second cord hole being positioned opposite each other across the diagonal slot;
the release cord traversing through the first cord hole and the second cord hole;
the release cord comprises a criss-cross portion and a pull portion;
the elastic locking band being positioned around the restrictor sleeve and the criss-cross portion opposite the flexible tube, wherein the release cord crosses over itself at the cross-cross portion, and wherein the release cord forms a loop around the diagonal slot at the criss-cross portion; and
the pull portion being positioned adjacent to the elastic locking band opposite the criss-cross portion.

12. The erection management system as claimed in claim 1 comprises:
the first pull handle and the second pull handle encircling the flexible tube.

13. The erection management system as claimed in claim 1 comprises:
the first lock washer being positioned between the first pull handle and the first end plug; and
the second lock washer being positioned between the second pull handle and the second end plug.

14. The erection management system as claimed in claim 1 comprises:
the first lock washer and the second lock washer being internal tooth lock washers;
the first lock washer encircling the flexible tube at the first end; and
the second lock washer encircling the flexible tube at the second end.

15. The erection management system as claimed in claim 1 comprises:
the first end plug and the second end plug being hollow.

16. The erection management system as claimed in claim 1 comprises:
the tension band assembly further comprises a comfort strip; and
the comfort strip being externally connected to the restrictor sleeve.

17. The erection management system as claimed in claim 1 comprises:
the tension band assembly further comprises a testicle band; and
the tension band being connected tangent to the tension loop opposite the restrictor sleeve.

18. The erection management system as claimed in claim 1 comprises:
the tension band assembly further comprises a sperm release cradle; and
the sperm release cradle being connected to the tension loop opposite the restrictor sleeve.
